(19) United States
(12) Patent Application Publication
(10) Pub. No.: US 2010/0178199 A1
(43) Pub. Date: Jul. 15, 2010

(54) Marine Air Purification System

(76) Inventor: Heath Schuman, Trappe, MD (US)

Correspondence Address: MEDLER FERRO PLLC, 8607 ROCKDALE LANE, SPRINGFIELD, VA, 22153, US

(21) Appl. No.: 12/353,900

(22) Filed: Jan. 14, 2009

(51) Int. Cl. A61L 2/10

(52) U.S. Cl. 422/24; 422/121

(57) ABSTRACT

An air purification unit for use in a boat includes a housing having an ultra-violet lamp capable of emitting electromagnetic radiation with a wavelength in the range of 315 nm to 400 nm, a titanium dioxide coating on an inside surface of the housing, and a titanium dioxide sleeve disposed over the lamp. Air entering the unit is purified by the hydroxyl radicals created by the reaction of the ultra-violet light and the titanium dioxide.

### BACKGROUND

1. Field of the Invention

The present invention relates generally to air purification systems for the boating industry and, in particular, to photo-catalytic oxidation air purification systems for the boating industry.

2. Related Art

Marine heating, ventilation, and air conditioning (HVAC) systems for use in the boating industry are well known. Air on boats is generally of rather poor quality, as engine rooms are often located adjacent or near indoor cabins and boats typically stay closed up without adequate ventilation for long periods of time. Thus, mold, mildew, and noxious odors accumulate in boats. There are generally dangerously high levels of mold spores and other contaminants in cabin air, creating an unhealthy atmosphere for people inside, especially those suffering from allergies.

Conventional marine HVAC systems utilize carbon filters and other conventional filters to attempt to clean the air circulated within the cabin. These systems do not sufficiently clean the air. Instead, conventional boat air conditioning systems merely move stale air around inside the boat without removing airborne contaminants.

Thus, there is a need for an improved a marine air purification system.

### SUMMARY OF THE INVENTION

The present invention solves the problem of poor quality air on boats by providing an air purification unit installed in a boat HVAC system. The air purification unit includes a housing, a titanium dioxide coating disposed on an interior surface of the housing, and a UV-A lamp disposed within the housing. In an embodiment, a titanium dioxide sleeve may be disposed over the lamp. In an embodiment, the titanium dioxide coating may include a first layer disposed adjacent to the interior surface of the housing and a second layer disposed on the first layer, wherein the second layer includes the titanium dioxide.

### BRIEF DESCRIPTION OF THE FIGURES

Embodiments of the present invention will now be described more fully with reference to the accompanying drawings in which like reference numerals indicate similar structure.

FIG. 1 is a schematic representation of an HVAC system for a boat including a purification system in accordance with an embodiment of the present invention.

FIG. 2 is schematic representation of the purification system of FIG. 1.

FIG. 3 is front view of the purification system of FIG. 1 with the front panel removed.

FIG. 4 is a bottom view of the purification system of FIG. 1, with the bottom panel and the ballast removed.

FIG. 5 is a cross-sectional view the UV lamp and sleeve taken along line A-A of FIG. 4.

FIG. 6 is a cross-section view of the housing and coating taken on line B-B of FIG. 4.

### DETAILED DESCRIPTION OF THE INVENTION

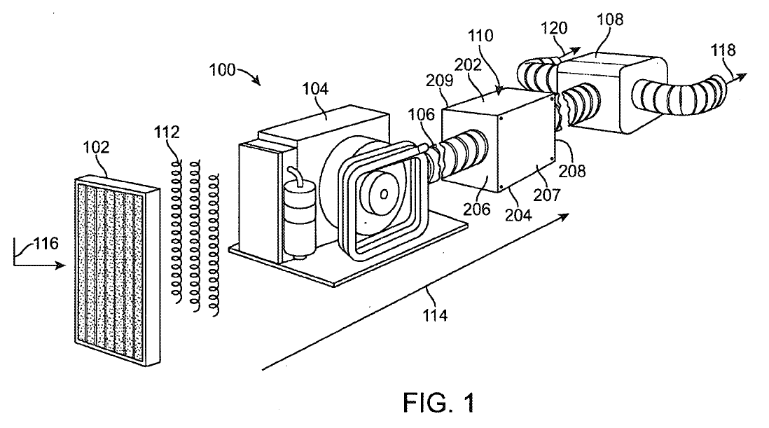

Referring now to FIG. 1, an HVAC system 100 for a boat is shown schematically. HVAC system 100 includes an air handling unit 104, duct 106, and an air purification system 110. HVAC system 100 may also include a distribution box 108 for distributing air to other areas of the boat. HVAC system 100 may also include a return air filter 102 and coils 112. Return air enters air handling unit 104 through return air filter 102 as shown at arrow 116. Although not shown, return air may return via return air ducts, similar to supply ducts 106. Alternatively, in smaller applications, a return air grille may be in a cabin and adjacent air handling unit 104 to return air directly to air handling unit 104 without the use of a return duct. Air passes over coils 112, through air handling unit 104 and into ducts 106, through air purification system 110, into distribution box 108, and out to rooms as shown by arrows 118, 120, as generally shown by arrows 114.

Air entering air handling unit 104 is either heated or cooled as it passes over coils 112, as known by those skilled in the art. Air handling unit 104 includes a compressor unit, a condenser, a blower, evaporator coils, and other parts known to those of ordinary skill in the art. The blower of air handling unit 104 distributes air throughout the boat through ducts 106. In the embodiment shown in FIG. 1, air purification system 110 is provided in supply ducts 106, but those skilled in the art would recognize that air purification system 100 could equally be provided in return ducts or in other locations in HVAC system. Similarly, distribution box 108 may not be needed in smaller applications, or several distribution boxes 108 may be needed in larger applications.

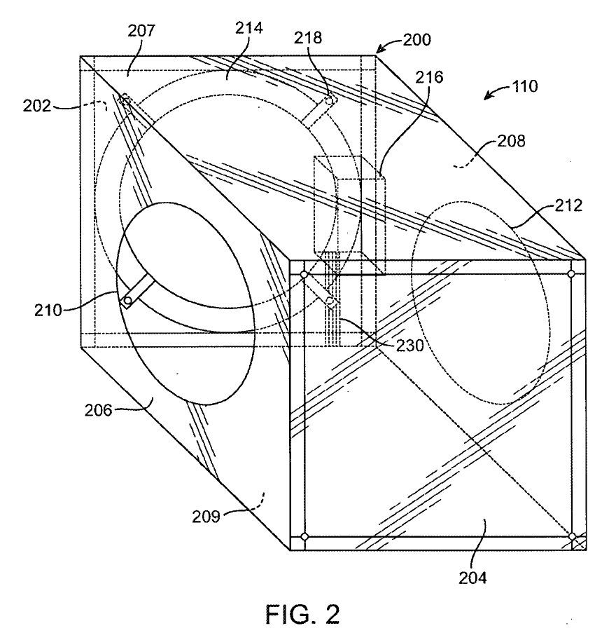

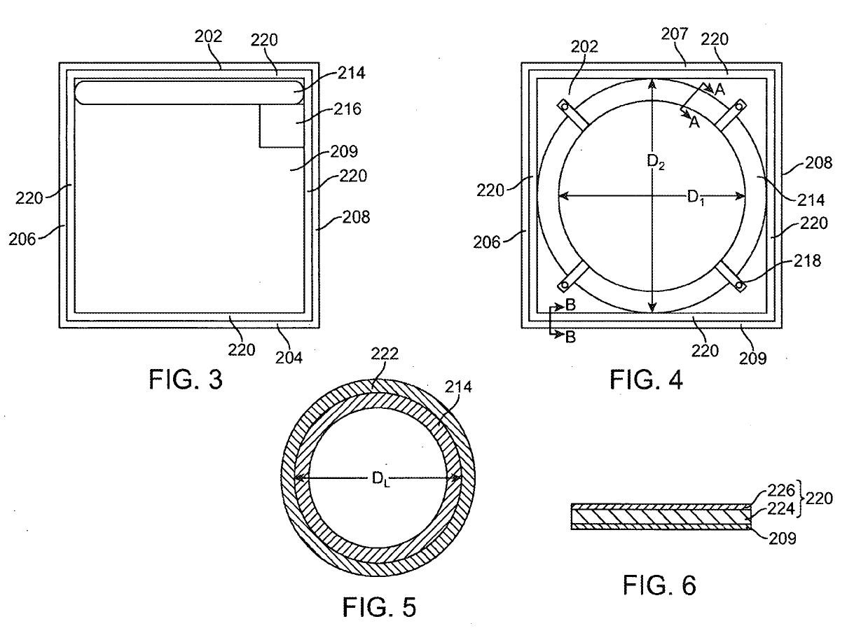

Air purification system 110 will now be described in greater detail with respect to FIGS. 2-6. FIG. 2 shows a schematic, perspective view of air purification system 110 with the panels of the housing depicted as see-through in order to show the interior of the system 110. Air purification system 110 includes a housing 200, including a top panel 202, a bottom panel 204, side panels 206, 208, a front panel 207 and a bottom panel 209. As would be understood by those of ordinary skill in the art, the labeling of the panels as top, bottom, front, back, or side is arbitrary depending on the location where air purification system is located, is used here for ease of description, and in no way is intended to limit the orientation or location of air purification system 110. Further, the terms used herein are used with respect to the schematic representation shown in FIG. 1 as if installed in a horizontal arrangement. FIG. 3 shows a front view air purification system 110 with front panel 207 removed. FIG. 4 shows a bottom view of air purification system 110 with bottom panel 204 removed.

As shown in FIG. 2, side panel 206 includes an opening 210 and side panel 208 includes an opening 212. Openings 210, 212 are provided to connect air purification system 110 to ducts 106, as shown in FIG. 1. Openings 210, 212 are round in the embodiment shown, but it would be understood by those skilled in the art that openings 210, 212 are shaped to accommodate the shape of ducts 206. Housing 200 is preferably made from shiny aluminum, but can also be made from aluminum, stainless steel, and other like materials known to those of ordinary skill in the art. Shiny aluminum is preferred because it reflects the light from the UV lamp, described in more detail below.

Disposed within housing 200 is an ultra-violet lamp (UV lamp) 214. UV lamp 214 emits electromagnetic radiation with a wavelength in the range of 315 nm to 400 nm (that is, UV-A light). UV lamp 214 is coupled to top panel 202 using straps 218 or other coupling devices. In the embodiment shown in FIG. 2, UV lamp 214 is a tube that is formed into a generally circular shape, which is preferred, although it may be other shapes as well. UV lamp 214 may be, for example, a 20 watt UV lamp. As shown in FIG. 5, a TiO₂ sleeve 222 is provided over UV lamp 214. Sleeve 222 may be for example, a glass fabric sleeve with TiO₂. UV lamp 214 with sleeve 222 may be, for example, model no. FCL22BL-TGT available from Taiwan Fluorescent Lamp Co., Ltd. (“TFC”) or similar lamps available from other manufacturers. In the embodiment shown, UV lamp 214 has an inner diameter D₁ of 150 mm, an outer diameter D₂ of 210 mm, and a lamp diameter D_{L} of 29 mm. One of ordinary skill in the art would recognize that different sized lamps may be used, depending on the size of the HVAC system 100 and the number of air purification systems 110 provided.

A ballast 216 is electrically connected to UV light 214. Ballast 216 may be, for example, a 20-40 watt electronic ballast, such as model no. EB-1005-03 available from Lightwave, or other similar ballasts available from other manufacturers. Electrical wiring 230 connects ballast 216 to a power source (not shown).

As shown in FIGS. 3, 4, and 6, the inner surface of housing 200 includes a coating 220. Coating 220 includes a primer layer 224 and a titanium dioxide (TiO₂) layer 226. Primer layer 224 is an encapsulating primer layer, such as NuTiO® photocatalytic pre-treatment encapsulating primer available from Bio Shield, Inc. of Port Richey, Fla. Primer layer 224 is sprayed on the interior surface of housing 200 first using a sprayer. This encapsulates odors and protects the aluminum housing from corrosion. Primer layer 224 also acts as a base coat to allow TiO₂ layer 226 to stick thereto. TiO₂ layer 226 is applied over primer layer 224 to ensure proper adhesion and longevity, and is preferably sprayed over primer layer 224 using a sprayer. Primer layer 224 and TiO₂ layer 226 are preferably clear, permitting the UV light to reflect off of the interior surface of the housing, which is preferably shiny aluminum, as discussed above.

TiO₂ layer 226 is preferably an all natural, un-doped 2.35% suspended colloid TiO₂ solution (percentage of TiO₂ suspended in water). As explained in more detail below, TiO₂ is a photo catalyst that reacts to the light from UV lamp 214 to create the decomposition of the cell membrane of contaminants such as mold, fungi, viruses, bacteria, VOC's, odors, etc. Using a TiO₂ solution with significantly less TiO₂ may result in incomplete decomposition of the cell membrane, thereby leading to an increase in formaldehyde, a possible by-product of conventional photocatalytic oxidation processes.

In the embodiment shown, duct 106 may be, for example, 7 inch diameter round duct and housing 200 may be, for example, and 9 inch by 9 inch by 9 inch cube. Further, housing 200 preferably does not include any baffles or diverters disposed therein. Such baffles or diverters normally act to direct air through a housing without turbulence. In the present invention, the size of housing 200 as larger than duct 106 and the shape of housing 200 with squared edges cause some turbulence in the air passing through housing 200. The size and turbulence thereby allow air passing through housing 200 additional time to be purified by the photocatalytic reaction described below.

In practice, when light energy the UV-A range emitted by UV lamp 214 hits anatase crystals of titanium oxide from coating 200 and sleeve 222, it turns the energized crystals into microscopic electron magnets. Water molecules pass by the energized TiO₂ electrons get pulled off the water molecule. This photocatalytic chemical reaction creates hydroxyl radicals as shown in the following equation.

TiO2+H2O+Air flow+UV light>TiOH+OH—+O2-(stable) (reactive)(reactive)

Thus, air entering housing 200 is exposed to hydroxyl radicals created by the reaction of UV lamp 214 with TiO₂ in coating 200 and sleeve 222. The hydroxyl radicals purify the air.

### EXAMPLE

An air purification system as described above was installed in a 48 foot VIKING motor yacht. The air purification system installed included a 9″ by 9″ by 9″ housing as described above connected to 7″ round duct. The 48 foot VIKING includes approximately 6,000 cubic feet of indoor air space volume. It further utilizes a 12,000 btu HVAC system that moves approximately 400 cubic feet of air per minute (cfm). One air purification system was installed in the supply side salon near the center of the boat. Prior to installation of the air purification system, a sample of air was captured for testing using a spore trap method. Testing of this pre-installation sample revealed mold spores in the amount of 1,834 spores per cubic meter of air. The air purification system was then installed. The HVAC system with the air purification system installed was run for 1 hour and a 1-hour sample was taken. Testing of the 1-hour sample revealed mold spores in the amount of 194 per cubic meter. Thus, in one hour, or approximately 4 air changes (400 cfm for 60 min is 24,000 cubic feet of air and the indoor air volume of the boat is approximately 6,000 cubic feet), the mold spore count was reduced by more than 89%. The HVAC system with the air purification system installed was run for another hour and a 2-hour sample was taken. Testing of the 2-hour sample revealed mold spores in the amount of 141 per cubic meter. The HVAC system with the air purification system installed was run for another hour and a 3-hour sample was taken. Testing of the 3-hour sample revealed mold spores in the amount of 206 per cubic meter.

The many features and advantages of the invention are apparent from the detailed specification and, thus, it is intended by the appended claims to cover all such features and advantages of the invention that fall within the true spirit and scope of the invention. Further, multiple embodiments have been described and different features of those embodiments may be interchanged. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. An air purification unit comprising:
a housing;
a coating disposed on an interior surface of the housing, wherein the coating comprises a first layer disposed adjacent to the interior surface and a second layer disposed on the first layer, wherein the second layer comprises titanium dioxide;
an ultra-violet lamp capable of emitting electromagnetic radiation with a wavelength in the range of 315 nm to 400 nm;
a sleeve disposed over the ultra-violet lamp, wherein the sleeve comprises titanium dioxide.

2. The air purification unit as claimed in claim 1, wherein said housing comprises shiny aluminum.

3. The air purification unit as claimed in claim 1, wherein said lamp is a tube formed into a generally circular shape.

4. The air purification unit as claimed in claim 1, further comprising an electronic ballast electrically coupled to the lamp.

5. An apparatus for removing contaminants from the air in a boat cabin, the apparatus comprising:
means for circulating air through the boat cabin; and
an air purification unit installed to receive a flow of circulating air, wherein the purification unit destroys contaminants in the circulating air, wherein the air purification unit includes a source of titanium dioxide and an ultra-violet light capable of emitting electromagnetic radiation with a wavelength in the range of 315 nm to 400 nm.

6. The apparatus as claimed in claim 5, wherein the air purification unit includes a housing and the source for titanium dioxide comprises a coating disposed on an inside surface of the housing.

7. The apparatus as claimed in claim 6, wherein the coating includes a first layer disposed adjacent to the interior surface and a second layer disposed on the first layer, wherein the second layer includes the titanium dioxide.

8. The apparatus as claimed in claim 6, wherein the source for titanium dioxide further comprises a titanium dioxide sleeve disposed over the ultra-violet lamp.

9. The apparatus as claimed in claim 5, wherein the source for titanium dioxide comprises a titanium dioxide sleeve disposed over the ultra-violet lamp.

10. A method for purifying air in a boat cabin comprising the steps of:
circulating air through the boat cabin; and
circulating the air through a housing, the housing including,
a titanium dioxide coating disposed on an inside surface of the housing, and
an ultra-violet light capable of emitting electromagnetic radiation with a wavelength in the range of 315 nm to 400 nm.

11. The method as claimed in claim 10, wherein titanium dioxide coating includes a first layer disposed adjacent to the interior surface and a second layer disposed on the first layer, wherein the second layer includes the titanium dioxide.

12. The method as claimed in claim 10, further comprising a titanium dioxide sleeve disposed over the ultra-violet lamp.

13. The method as claimed in claim 10, wherein more than 80% of contaminants are removed from the air in boat cabin.

14. The method as claimed in claim 14, wherein the contaminants are removed in four air exchanges.
